(19) United States
(12) Patent Application Publication
(10) Pub. No.: US 2024/0398403 A1
(43) Pub. Date: Dec. 5, 2024

(54) SUTURE ASSEMBLY

(71) Applicant: Raymond Michael Greiwe, Edgewood, KY (US)

(72) Inventor: Raymond Michael Greiwe, Edgewood, KY (US)

(21) Appl. No.: 18/699,111

(22) PCT Filed: Oct. 5, 2022
(86) PCT No.: PCT/US2022/045743

§ 371 Date: Apr. 5, 2024

Related U.S. Application Data
(60) Provisional application No. 63/252,278, filed on Oct. 5, 2021.

(51) Int. Cl. A61B 17/04

(52) U.S. Cl. CPC A61B 17/0401; A61B 2017/0414; A61B 2017/042; A61B 2017/0446

(57) ABSTRACT

A suture assembly includes a first strand having a first end, and a second end, the first strand configured to be secured to one or more suture anchors, a second strand having a third end, a fourth end, the second strand configured to be secured to the one or more suture anchors, a third strand coupled to first strand at a first intersection and the second strand at a second intersection, a fourth strand coupled to the first strand at a third intersection and the second strand at a fourth intersection, and a mesh attached to and extending between the first strand, the second strand, the third strand and the fourth strand.

### CROSS-REFERENCES TO RELATED APPLICATIONS

This application claims the benefit of priority to U.S. Provisional Patent Application No. 63/252,278, filed on Oct. 5, 2021, which is hereby incorporated by reference herein in its entirety for all purposes.

### FIELD

The present disclosure relates to apparatuses, systems, and methods for surgery. More particularly, the present disclosure relate to apparatuses, systems, and methods for securing a suture assembly at a surgical site.

### BACKGROUND

Sutures may be used to facilitate wound closure and optimize healing conditions. Further, sutures may provide mechanical support to facilitate closure of the wound. For example, a suture anchor may be inserted into a surgical site and a suture strands may be coupled to the anchor. The suture strand may then be coupled to a portion of tissue to secure that tissue to the suture anchor via the suture strand.

### SUMMARY

According to an aspect of the present disclosure, a suture assembly includes a first strand having a first end, and a second end, the first strand configured to be secured to one or more suture anchors, a second strand having a third end, a fourth end, the second strand configured to be secured to the one or more suture anchors, a third strand coupled to first strand at a first intersection and the second strand at a second intersection, a fourth strand coupled to the first strand at a third intersection and the second strand at a fourth intersection, and a mesh attached to and extending between the first strand, the second strand, the third strand and the fourth strand.

According to another aspect of the present disclosure, a method of securing tissue includes inserting a first suture anchor at a first location of a surgical site and a second suture anchor at a second location of the surgical site, positioning a suture assembly at the surgical site, the suture assembly comprising a first strand, a second strand, a third strand, a fourth strand, and a mesh attached to and extending between the first strand, the second strand, the third strand and the fourth strand, wherein the third strand and the fourth strand are adjustably engaged with a first portion and a second portion of the first strand, respectively, and the third strand and the fourth strand are adjustably engaged with a third portion and a fourth portion of the second strand, respectively, attaching at least one of a first end of the first strand or a third end of the second strand to the first suture anchor, attaching at least one of a second end of the first strand or a fourth end of the second strand to the second suture anchor, and adjusting the mesh relative to the surgical site by adjusting at least one of a first tension in the first strand or a second tension in the third strand to cause a change in a third tension in the third strand and a fourth tension in the fourth strand.

According to another aspect of the present disclosure, a surgical kit includes a first suture anchor, a second suture anchor, and a suture assembly configured to be coupled to the first suture anchor and the second suture anchor. The suture assembly includes a first strand having a first end, and a second end, the first strand configured to be secured to one or more suture anchors, a second strand having a third end, a fourth end, the second strand configured to be secured to the one or more suture anchors, a third strand coupled to first strand at a first intersection and the second strand at a second intersection, a fourth strand coupled to the first strand at a third intersection and the second strand at a fourth intersection, and a mesh attached to and extending between the first strand, the second strand, the third strand and the fourth strand.

This summary is illustrative only and should not be regarded as limiting.

### BRIEF DESCRIPTION OF THE DRAWINGS

FIG. 1 is a top view of a suture assembly, according to an example embodiment.

FIG. 2 is a top view of another suture assembly, according to an example embodiment.

FIG. 3 is a top view of another suture assembly, according to an example embodiment.

FIG. 4 is a top view of another suture assembly, according to an example embodiment.

FIG. 5 is a top view of another suture assembly, according to an example embodiment.

FIG. 6 is a cross sectional view of a suture anchor in a first orientation, according to an example embodiment.

FIG. 7 is a cross sectional view of the suture anchor of FIG. 6 in a second orientation, according to an example embodiment.

FIG. 8 is a perspective view of a surgical site, according to an example embodiment.

FIG. 9 is a perspective view of the suture assembly of FIG. 2 implanted at the surgical site of FIG. 8.

FIG. 10 is a perspective view of a surgical site, according to an example embodiment.

FIG. 11 is a perspective view of the suture assembly of FIG. 5 implanted at the surgical site of FIG. 10.

FIG. 12 is a flow diagram of a method of securing tissue, according to an example embodiment.

FIG. 13 is a cross sectional view of another suture anchor, according to an example embodiment.

FIG. 14 is another cross sectional view of the suture anchor of FIG. 13, according to an example embodiment.

FIG. 15 is a cross sectional view of another suture anchor, according to an example embodiment.

FIG. 16 is another cross sectional view of the suture anchor of FIG. 15, according to an example embodiment.

FIGS. 17-19 illustrate the process of tying a non-slipping knot for use within the suture anchor of FIG. 15, according to an example embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

The use of “e.g.” “etc.,” “for instance,” “in example,” and “or” and grammatically related terms indicates non-exclusive alternatives without limitation, unless otherwise noted. The use of “optionally” and grammatically related terms means that the subsequently described element, event, feature, or circumstance may or may not be present/occur, and that the description includes instances where said element, event, feature, or circumstance occurs and instances where it does not. The use of “attached” and “coupled” and grammatically related terms refers to the fixed, releasable, or integrated association of two or more elements and/or devices with or without one or more other elements in between. Thus, the term “attached” or “coupled” and grammatically related terms include releasably attaching or fixedly attaching two or more elements and/or devices in the presence or absence of one or more other elements in between. As used herein, the terms “proximal” and “distal” are used to describe opposing axial ends of the particular elements or features being described in relation to anatomical placement.

Medical sutures may be used to facilitate closing wounds to skin or other tissue (e.g., tendons, muscle tissue, cartilage, connective tissue, epithelial tissue, nervous tissue, etc.). For example, a medical professional may use a needle attached to a suture to stitch together a wound to a patient's epithelial tissue. Further, a medical professional may use suture to attach muscle tissue or connective tissue to a solid structure (e.g., one or more bones) in the patient. For example, suture may be used to reattach rotator cuff tendons to the head of the humerus as a part of a rotator cuff repair surgery. During the rotator cuff repair surgery, a surgeon may implant a first suture anchor into the head of the humerus, couple a suture strand to the suture anchor, thread the suture strand through the rotator cuff tendon, and couple the suture strand to a second anchor implanted into the head of the humerus to secure the rotator cuff tendons in a desired location such that rotator cuff tendons can reattach to the head of the humerus. In this example, the suture strand may be a single strand of material or multiple strands of fabric braided into a single strand. Further, the rotator cuff tendon surgery described above may require multiple suture strands to properly secure the rotator cuff tendon. In this case, the surgeon needs to take caution to avoid intertwining the multiple suture strands so that the multiple suture strands do not become knotted and to ensure that the multiple suture strands remain in tension to retain the rotator cuff tendons. It should be appreciated that, while reference is repeatedly made herein to rotator cuff tendon surgery, the embodiments and methods described herein may be utilized in a number of different medical procedures including, but not limited to, bicep reattachment surgery, Achilles reattachment surgery, reattachment of various tendons in a person's hands or feet, and/or any type of medical procedure that utilizes suture strands.

Referring now to the Figures generally, a suture assembly is shown, according to an example embodiment. The suture assembly includes a first strand having a first end, and a second end and is configured to be secured to one or more suture anchors. The suture assembly further includes a second strand having a third end, a fourth end, and configured to be secured to the one or more suture anchors. The suture assembly further includes a third strand coupled to first strand at a first intersection and the second strand at a second intersection and a fourth strand coupled to the first strand at a third intersection and the second strand at a fourth intersection. Further, the suture assembly includes a mesh attached to and extending between the first strand, the second strand, the third strand and the fourth strand. Utilizing a suture assembly described herein may provide several advantages over using individual suture strands. For example, according to various embodiments, the suture assemblies described herein may reduce the time and effort required for a desired surgery. For example, since the strands are coupled to one another, each strand does not necessarily need to be coupled to one or more suture anchors. Further, the individual strands are less likely to become tangled with one another during the surgery. Further, the mesh of the suture assembly may cover at least a portion of the wounded area, and therefore improve and accelerate healing. While a surgeon may utilize mesh in conjunction with individual strands of suture, the mesh may be prone to folding, creasing, or otherwise not bunching up as the mesh is not coupled to the individual suture strands. Thus, coupling the mesh to the multiple suture strands reduces the risk of the mesh bunching up, thereby improving the performance of the mesh. Thus, for these reasons and the other reasons described herein, the suture assemblies described herein provide a distinct advantage over using individual strands of suture that are not coupled to one another.

According to various embodiments, the first strand comprises a first tapered portion proximate the first end and a second tapered portion proximate the second end. The first tapered portion and the second tapered portion may facilitate coupling the suture assembly to one or more suture anchors. For example, the tapered ends may facilitate threading the strands into a guide loop of a suture anchor to couple the strand to the suture anchor. According to various embodiments, the first strand is substantially perpendicular to the third strand proximate the first intersection and the second strand is substantially perpendicular to the second strand proximate the second intersection. This orientation may reduce the risk of the mesh coupled to the first strand and the third strand from bunching up. According to various embodiments, the mesh comprises suture tape. According to various embodiments, the mesh comprises aligned polymer fibers (e.g., Poly Lactic-co-Glycolic Acid biodegradable fibers). According to various embodiments, the first strand comprises a first plurality of strands, the second strand comprises a second plurality of strands, the third strand includes a third plurality of strands, and the fourth strand includes a fourth plurality of strand, and the first plurality of strands is interwoven with the third plurality of strands and the fourth plurality of strands, and the second plurality of strands is interwoven with the third plurality of strands and the fourth plurality of strands. By interweaving the strands, the suture assembly may have increased strength and be prevented from coming apart when used. According to various embodiments, the first plurality of strands is woven in a front-back orientation. This weave pattern may cause the interwoven strands to tighten around one another when the tension in the first strand is increased, thereby increasing compression on the surgical site. According to various embodiments, the first strand extends in a first direction between the first intersection and the third intersection and the third strand extends in a second direction between the first intersection and the second intersection, and increasing a first tension in the first direction in the first strand causes an increase in a second tension in the second direction in the second strand. This allows tension in the third strand and the fourth strand to be adjusted using the first strand and/or the second strand.

According to various embodiments, method of securing tissue is disclosed according to an example embodiment. The method includes inserting a first suture anchor at a first location of a surgical site and a second suture anchor at a second location of the surgical site, positioning a suture assembly at the surgical site, the suture assembly comprising a first strand, a second strand, a third strand, a fourth strand, and a mesh attached to and extending between the first strand, the second strand, the third strand and the fourth strand, wherein the third strand and the fourth strand are adjustably engaged with a first portion and a second portion of the first strand, respectively, and the third strand and the fourth strand are adjustably engaged with a third portion and a fourth portion of the second strand, respectively, attaching at least one of a first end of the first strand or a third end of the second strand to the first suture anchor, attaching at least one of a second end of the first strand or a fourth end of the second strand to the second suture anchor, and adjusting the mesh relative to the surgical site by adjusting at least one of a first tension in the first strand or a second tension in the third strand to cause a change in a third tension in the third strand and a fourth tension in the fourth strand.

According to various embodiments, the method may further include inserting a third suture anchor at a third location of the surgical site and attaching at least one of the first end of the first strand or the third end of the second strand to the third suture anchor. The method may further include inserting a fourth suture anchor at a fourth location of the surgical site, and attaching at least one of the second end of the first strand or the fourth end of the second strand to the fourth suture anchor. According to various embodiments, the first strand comprises a first tapered portion proximate the first end and a second tapered portion proximate the second end. According to various embodiments, coupling the first end of the first strand to the first suture anchor comprises inserting the first tapered portion into a guide loop in the first suture anchor. According to various embodiments, the first strand and the second strand are substantially perpendicular to the third strand and the fourth strand proximate the mesh. According to various embodiments, the mesh comprises suture tape. According to various embodiments, the mesh comprises individual strands of suture. According to various embodiments, the mesh comprises aligned polymer fibers, such as Poly Lactic-co-Glycolic Acid or any other biodegradable fibers. According to various embodiments, the mesh comprises collagen polymer fibers. According to various embodiments, the mesh may promote collagen proliferation proximate the surgical site, which may improve healing.

According to various embodiments, the first strand comprises a first plurality of strands, the second strand comprises a second plurality of strands, the third strand includes a third plurality of strands, and the fourth strand includes a fourth plurality of strands, wherein the first plurality of strands is interwoven with the third plurality of strands and the fourth plurality of strands, and wherein the second plurality of strands is interwoven with the third plurality of strands and the fourth plurality of strands. According to various embodiments, the first plurality of strands is woven in a front-back orientation.

According to various embodiments, a surgical kit is disclosed. The surgical kit includes a first suture anchor, a second suture anchor, and a suture assembly configured to be coupled to the first suture anchor and the second suture anchor. The suture assembly includes a first strand having a first end, and a second end, the first strand configured to be secured to one or more suture anchors, a second strand having a third end, a fourth end, the second strand configured to be secured to the one or more suture anchors, a third strand coupled to first strand at a first intersection and the second strand at a second intersection, a fourth strand coupled to the first strand at a third intersection and the second strand at a fourth intersection, and a mesh attached to and extending between the first strand, the second strand, the third strand and the fourth strand.

According to various embodiments, the first strand comprises a first tapered portion proximate the first end and a second tapered portion proximate the second end. According to various embodiments, the first strand and the second strand are substantially perpendicular to the third strand and the fourth strand proximate the mesh. According to various embodiments, the mesh comprises suture tape. According to various embodiments, the mesh comprises individual strands of suture. According to various embodiments, the mesh comprises aligned polymer fibers, such as Poly Lactic-co-Glycolic Acid or any other biodegradable fibers. According to various embodiments, the mesh comprises collagen polymer fibers. According to various embodiments, the first suture anchor and the second suture anchor are knotless self-punching shuttle anchors. According to various embodiments, the first strand comprises a first plurality of strands, the second strand comprises a second plurality of strands, the third strand includes a third plurality of strands, and the fourth strand includes a fourth plurality of strands, wherein the first plurality of strands is interwoven with the third plurality of strands and the fourth plurality of strands, and wherein the second plurality of strands is interwoven with the third plurality of strands and the fourth plurality of strands. According to various embodiments, the first plurality of strands is woven in a front-back orientation. According to various embodiments, the first strand extends in a first direction between the first intersection and the third intersection and the third strand extends in a second direction between the first intersection and the second intersection, and increasing a first tension in the first direction in the first strand causes an increase in a second tension in the second direction.

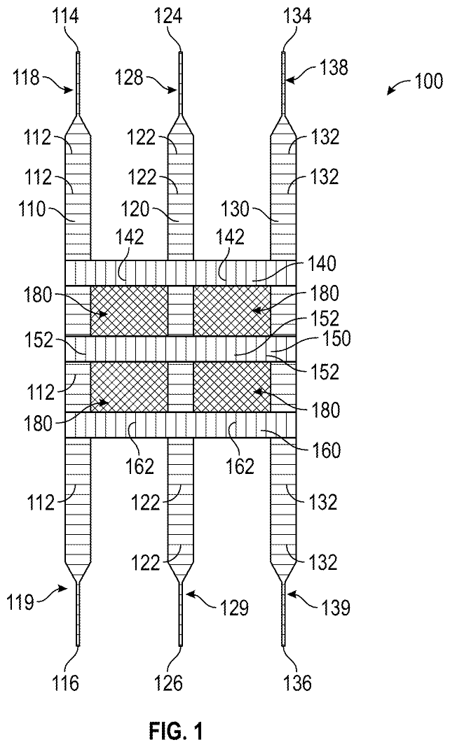

Referring now to FIG. 1, a top view of a suture assembly 100, according to an example embodiment. As is described further herein, the suture assembly 100 may be used to secure a portion of tissue in a desired location of a surgical site. As shown, the suture assembly 100 includes a first strand 110, a second strand 120, and a third strand 130. As shown, the first strand 110 extends in a first direction, the second strand 120 extends in a second direction, and the third strand 130 runs in a third direction, wherein the first direction, the second direction, and the third direction are all substantially parallel (e.g., within 2 degrees) with one another. As shown, the first strand 110 includes a first tapered portion 118 proximate a first end 114 and a second tapered portion 119 proximate a second end 116 of the first strand 110. As is discussed further herein, the tapered portions 118, 119 may facilitate coupling the first strand 110 to a suture anchor (e.g., by enabling the first end 114 or the second end 116 to be threaded through a guide loop in a suture anchor). Similarly, the second strand 120 includes a first tapered portion 128 proximate a first end 124 and a second tapered portion 129 proximate a second end 126 of the second strand 120. Similarly, the third strand 130 includes a first tapered portion 138 and proximate a first end 134 and a second tapered portion 139 proximate a second end 136.

The suture assembly 100 further includes a fourth strand 140, a fifth strand 150, and a sixth strand 160. As shown, the fourth strand 140 is coupled to the first strand 110 at a first intersection, the second strand 120 at a second intersection, and the third strand 130 at a third intersection. Similarly, the fifth strand 150 is coupled to the first strand 110 at a fourth intersection, the second strand 120 and a fifth intersection, and the third strand 130 at a sixth intersection. Similarly, the sixth strand 160 is coupled to the first strand 110 at a seventh intersection, the second strand 120 and an eighth intersection, and the third strand 130 at a ninth intersection. As shown, the fourth strand 140 extends in a fourth direction, the fifth strand 150 extends in a fifth direction, and the sixth strand 160 extends in a sixth direction, wherein the fourth direction, the fifth direction, and the sixth direction are all substantially parallel (e.g., within 2 degrees) to one another. Further, the first strand 110, the second strand 120, and the third strand 130 are substantially perpendicular (e.g., within 2 degrees) to the fourth strand 140, the fifth strand 150, and the sixth strand 160 proximate the respective intersections (e.g., the first intersection, the second intersection, etc.).

According to various embodiments, the first strand 110 includes a first plurality of strands 112, the second strand 120 includes a first plurality of strands 122, the third strand 130 may include a third plurality of strands 132, the fourth strand 140 includes a fourth plurality of strands 142, the fifth strand 150 may include a fifth plurality of strands 152, and the sixth strand 160 includes a sixth plurality of strands 162. For example, each of the plurality of strands may include individual strands of absorbable sutures (e.g., gut, Polydioxanone (PDS), Poliglecaprone (MONOCRYL), Polyglactin (Vicryl), etc.) and/or nonabsorbable sutures (e.g., nylon, Polypropylene (Prolene), silk, Polyester (Ethibond), etc.). According to various embodiments, each of the plurality of strands may be braided (e.g., cylindrical wound braid, helically wound braid, etc.) or woven (e.g., front-back, plain, twill, satin, etc.) together into a single strand. For example, the first plurality of strands 112 may be woven in a front-back pattern weave to create the first strand 110.

According to various embodiments, each of the strands may be interwoven with any or all of the other strands. For example, the fourth strand 140 may be interwoven with the first strand 110 at the first intersection, the second strand 120 at the second intersection, and/or the third strand 130 at the third intersection. In this sense, one or more of the fourth plurality of strands 142 may be the same as the first plurality of strands 112, the second plurality of strands 122, and/or the third plurality of strands 132. In other words, one of the individual strands of the first plurality of strands 112 may be the same as one of the individual strands in any of the other plurality of strands 122, 132, 142, 152, 162. Thus, the first strand 110, the second strand 120, the third strand 130, the fourth strand 140, the fifth strand 150, and the sixth strand 160 may all share one or more individual strands, thereby coupling each of the strands together.

According to various embodiments, the braid or weave pattern may cause the interwoven strands to tighten around one another when the tension in an individual strand is increased. For example, increasing tension in the first strand 110 may cause each of the plurality of strands 112 to tighten around one another. Further, in the example embodiment where one or more of the plurality of strands 112 is interwoven into the third strand 130, increasing tension in the first strand 110 may also increase tension in the fourth strand 140 as a result of the individual strands shared by both the first strand 110 and the fourth strand. In this example embodiment, increasing tension in the first strand 110 will increase tension in the fourth strand 140. If the suture assembly is installed into a rounded (e.g., convex) surgical site, increasing the tension in the fourth strand 140 may increase compression on the surgical site that the suture assembly 100 is installed into. According to various embodiments, increasing the compression may facilitate healing of the wound. It should be appreciated that the tension in the fifth strand 150 and the sixth strand 160 may be increased in a similar fashion. Further, increasing the tension in the second strand 120 and/or the third strand 130 may similarly increase the tension in the fourth strand 140, the fifth strand 150, and the sixth strand 160.

As shown, the suture assembly 100 further includes a mesh 180 coupled to and extending between the first strand 110, the second strand 120, the third strand 130, the fourth strand 140, the fifth strand 150, and the sixth strand 160. Since the mesh 180 is coupled to each of the strands, the mesh 180 is less likely to bunch up once each of the strands are secured to a suture anchor because the mesh 180 will remain taut once the strands and suture anchors are secured, as is discussed further below. According to various embodiments, the mesh 180 may include a plurality of strands or fibers. For example, the mesh 180 may include a plurality of aligned polymer fibers, such as Poly Lactic-co-Glycolic Acid biodegradable fibers or any other biodegradable fibers. According to various embodiments, the mesh comprises aligned collagen polymer fibers. According to various embodiments, the mesh may promote collagen proliferation proximate the surgical site, which may improve healing.

According to various embodiments, the strands or fibers of the mesh 180 may be interwoven into one or more of the first strand 110, the second strand 120, the third strand 130, the fourth strand 140, the fifth strand 150, and the sixth strand 160. In this sense, increasing the tension in any of the strands may also increase the tension in the mesh 180, which may result in greater compression at the surgical site, in a similar manner as described above.

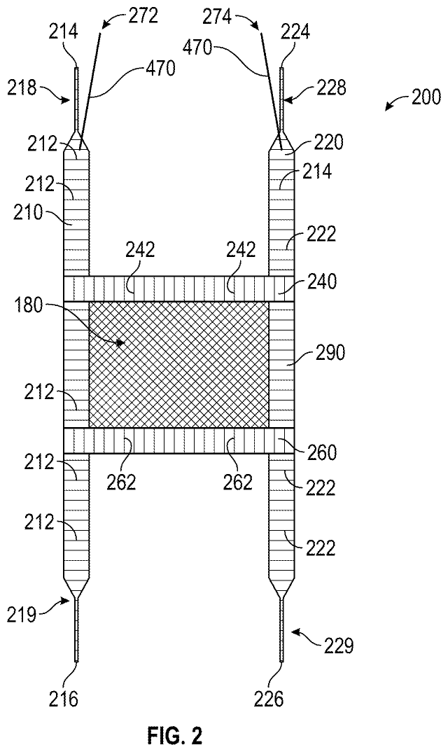

Referring now to FIG. 2, a top view of another suture assembly 200, according to an example embodiment. The suture assembly 200 may be similar to the suture assembly 100 described above. As is described further herein, the suture assembly 200 may be used to secure a portion of tissue in a desired location of a surgical site. As shown, the suture assembly 200 includes a first strand 210, and a second strand 220. As shown, the first strand 210 extends in a first direction and the second strand 220 extends in a second direction, wherein the first direction and the second direction are substantially parallel (e.g., within 2 degrees) with one another. As shown, the first strand 210 includes a first tapered portion 218 proximate a first end 214 and a second tapered portion 219 proximate a second end 216 of the first strand 210. As is discussed further herein, the tapered portions 218, 219 may facilitate coupling the first strand 210 to a suture anchor (e.g., by enabling the first end 214 or the second end 216 to be threaded through a guide loop in a suture anchor). Similarly, the second strand 220 includes a first tapered portion 228 proximate a first end 224 and a second tapered portion 229 proximate a second end 226 of the second strand 220.

The suture assembly 200 further includes a third strand 240 and a fourth strand 260. As shown, the third strand 240 is coupled to the first strand 210 at a first intersection and the second strand 220 at a second intersection. Similarly, the fourth strand 260 is coupled to the first strand 210 at a third intersection and the second strand 220 and a fourth intersection. As shown, the third strand 240 extends in a third direction and the fourth strand 260 extends in a fourth direction, wherein the third direction and the fourth direction are substantially parallel (e.g., within 2 degrees) to one another. Further, the first strand 210 and the second strand 220 are substantially perpendicular (e.g., within 2 degrees) to the third strand 240 and the fourth strand 260 proximate the respective intersections (e.g., the first intersection, the second intersection, etc.).

According to various embodiments, the first strand 210 includes a first plurality of strands 212, the second strand 220 includes a first plurality of strands 222, the third strand 240 may include a third plurality of strands 242, and the fourth strand 260 includes a fourth plurality of strands 262. For example, each of the plurality of strands may include individual strands of absorbable sutures (e.g., gut, Polydioxanone (PDS), Poliglecaprone (MONOCRYL), Polyglactin (Vicryl), etc.) and/or nonabsorbable sutures (e.g., nylon, Polypropylene (Prolene), silk, Polyester (Ethibond), etc.). According to various embodiments, each of the plurality of strands may be braided (e.g., cylindrical wound braid, helically wound braid, etc.) or woven (e.g., front-back, plain, twill, satin, etc.) together into a single strand. For example, the first plurality of strands 212 may be woven in a front-back pattern weave to create the first strand 210.

According to various embodiments, each of the strands may be interwoven with any or all of the other strands. For example, the fourth strand 260 may be interwoven with the first strand 210 at the third intersection and the second strand 220 at the fourth intersection. In this sense, one or more of the fourth plurality of strands 262 may be the same as one or more of the first plurality of strands 212, the second plurality of strands 222, and/or the third plurality of strands 242. In other words, one of the individual strands of the first plurality of strands 212 may be the same as one of the individual strands in any of the other plurality of strands 222, 242, 262. Thus, the first strand 210, the second strand 220, the third strand 240, and the fourth strand 260 may all share one or more individual strands, thereby coupling each of the strands together.

According to various embodiments, the braid or weave pattern may cause the interwoven strands to tighten around one another when the tension in an individual strand is increased. For example, increasing tension in the first strand 210 may cause each of the plurality of strands 212 to tighten around one another. Further, in the example embodiment where one or more of the plurality of strands 212 is interwoven into the third strand 240, increasing tension in the first strand 210 may also increase tension in the fourth strand 260 as a result of the individual strands shared by both the first strand 210 and the fourth strand. In this example embodiment, increasing tension in the first strand 210 will increase tension in the fourth strand 260. If the suture assembly is installed into a rounded (e.g., convex) surgical site, increasing the tension in the fourth strand 260 may increase compression on the surgical site that the suture assembly 200 is installed into. According to various embodiments, increasing the compression may facilitate healing of the wound.

As shown, the suture assembly 200 further includes a mesh 280 coupled to and extending between the first strand 210, the second strand 220, the third strand 240, and the fourth strand 260. Since the mesh 280 is coupled to each of the strands, the mesh 280 is less likely to bunch up once each of the strands are secured to a suture anchor because the mesh 280 will remain taut once the strands and suture anchors are secured, as is discussed further below. According to various embodiments, the mesh 280 may include a plurality of strands or fibers. For example, the mesh 280 may include a plurality of aligned polymer fibers, such as Poly Lactic-co-Glycolic Acid biodegradable fibers or any other type of biodegradable fibers. According to various embodiments, the mesh comprises collagen polymer fibers. According to various embodiments, the mesh may promote collagen proliferation proximate the surgical site, which may improve healing.

According to various embodiments, the strands or fibers of the mesh 280 may be interwoven into one or more of the first strand 210, the second strand 220, the third strand 240, and/or the fourth strand 260. In this sense, increasing the tension in any of the strands may also increase the tension in the mesh 280, which may result in greater compression at the surgical site, in a similar manner as described above.

According to various embodiments, the suture assembly 200 further includes one or more securing strands 270. As is discussed further herein, the securing strand 270 may be secured to one or more suture anchors to increase the compressive force exerted by the suture assembly 200. For example, as shown, the suture assembly 200 includes a securing strand 270 having a first end 272 and a second end 274. As shown, the securing strand 270 is coupled to the remainder of the suture assembly 200. For example, the first strand 210, the second strand 220, and the third strand 240 may be woven such that the each strand defines an inner volume that the securing strand 270 can be threaded through. For example, the first strand 210, the second strand 220, and the third strand 240 may be woven such that the inner volume of each strand is connected. As shown, the securing strand 270 starts at the first end 272, enters the internal volume of the first strand 210, continues through the internal volume of the third strand 240, continues through the internal volume of the second strand 220, and the second end 274 extends out of the second strand 220.

The first end 272 and the second end 274 of the securing strand 270 are configured to be coupled to a suture anchor. For example, the first end 272 may be coupled to the same suture anchor as the first end 214 of the first strand 210 and the second end 274 may be coupled to the same suture anchor as the first end 224 of the second strand 220. Tension in the securing strand 270 may then be increased by pulling the first end 272 and/or the second end 274 through the respective suture anchor. By increasing the tension in the securing strand 270, the tension in the first strand 210, the second strand 220, and the third strand 240 may also increase, thereby increasing the compressive force of the suture assembly 200 (e.g., proximate the point the securing strand 270 is coupled to the suture assembly 200) at the surgical site. It should be appreciated that, while the suture assembly 200 is shown to include just one securing strand 270, other embodiments may include a plurality of securing strand 270. For example, additional securing strands 270 may be threaded into an internal volume of the first strand 210, the fourth strand 260, and the second strand 220 in a similar fashion.

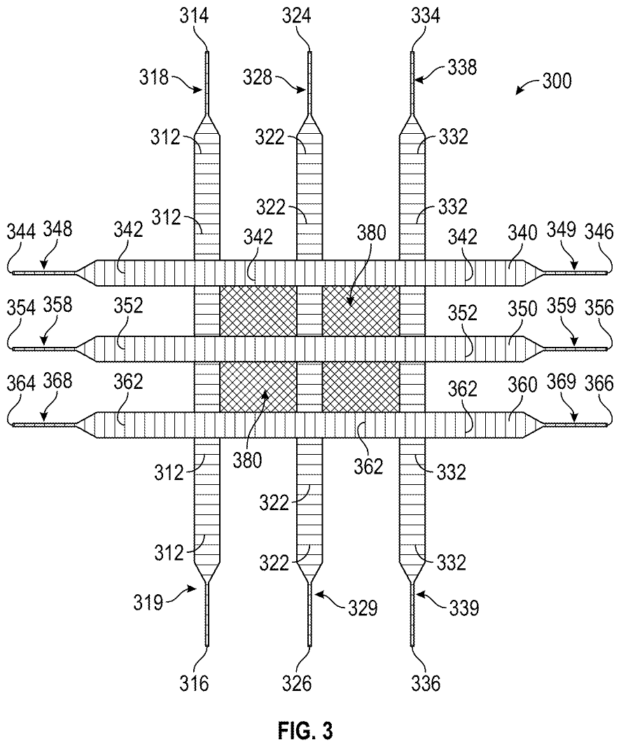

Referring now to FIG. 3, a top view of another suture assembly 300, according to an example embodiment. The suture assembly 300 may be similar to the suture assemblies 100, 200 described above. As is described further herein, the suture assembly 300 may be used to secure a portion of tissue in a desired location of a surgical site. As shown, the suture assembly 300 includes a first strand 310, a second strand 320, and a third strand 330. As shown, the first strand 310 extends in a first direction, the second strand 320 extends in a second direction, and the third strand 330 runs in a third direction, wherein the first direction, the second direction, and the third direction are all substantially parallel (e.g., within 2 degrees) with one another. As shown, the first strand 310 includes a first tapered portion 318 proximate a first end 314 and a second tapered portion 319 proximate a second end 316 of the first strand 310. As is discussed further herein, the tapered portions 318, 319 may facilitate coupling the first strand 310 to a suture anchor (e.g., by enabling the first end 314 or the second end 316 to be threaded through a guide loop in a suture anchor). Similarly, the second strand 320 includes a first tapered portion 328 proximate a first end 324 and a second tapered portion 329 proximate a second end 326 of the second strand 320. Similarly, the third strand 330 includes a first tapered portion 338 and proximate a first end 334 and a second tapered portion 339 proximate a second end 336.

The suture assembly 300 further includes a fourth strand 340, a fifth strand 350, and a sixth strand 360. As shown, the fourth strand 340 includes a first tapered portion 348 proximate a first end 344 and a second tapered portion 349 proximate a second end 346 of the fourth strand 340. As is discussed further herein, the tapered portions 348, 349 may facilitate coupling the fourth strand 340 to a suture anchor (e.g., by enabling the first end 344 or the second end 346 to be threaded through a guide loop in a suture anchor). Similarly, the fifth strand 350 includes a first tapered portion 358 proximate a first end 354 and a second tapered portion 359 proximate a second end 356 of the fifth strand 350. Similarly, the sixth strand 360 includes a first tapered portion 368 and proximate a first end 364 and a second tapered portion 369 proximate a second end 366.

As shown, the fourth strand 340 is coupled to the first strand 310 at a first intersection, the second strand 320 at a second intersection, and the third strand 330 at a third intersection. Similarly, the fifth strand 350 is coupled to the first strand 310 at a fourth intersection, the second strand 320 and a fifth intersection, and the third strand 330 at a sixth intersection. Similarly, the sixth strand 360 is coupled to the first strand 310 at a seventh intersection, the second strand 320 and an eighth intersection, and the third strand 330 at a ninth intersection. As shown, the fourth strand 340 extends in a fourth direction, the fifth strand 350 extends in a fifth direction, and the sixth strand 360 extends in a sixth direction, wherein the fourth direction, the fifth direction, and the sixth direction are all substantially parallel (e.g., within 2 degrees) to one another. Further, the first strand 310, the second strand 320, and the third strand 330 are substantially perpendicular (e.g., within 2 degrees) to the fourth strand 340, the fifth strand 350, and the sixth strand 360 proximate the respective intersections (e.g., the first intersection, the second intersection, etc.).

According to various embodiments, the first strand 310 includes a first plurality of strands 312, the second strand 320 includes a first plurality of strands 322, the third strand 330 may include a third plurality of strands 332, the fourth strand 340 includes a fourth plurality of strands 342, the fifth strand 350 may include a fifth plurality of strands 352, and the sixth strand 360 includes a sixth plurality of strands 362. For example, each of the plurality of strands may include individual strands of absorbable sutures (e.g., gut, Polydioxanone (PDS), Poliglecaprone (MONOCRYL), Polyglactin (Vicryl), etc.) and/or nonabsorbable sutures (e.g., nylon, Polypropylene (Prolene), silk, Polyester (Ethibond), etc.). According to various embodiments, each of the plurality of strands may be braided (e.g., cylindrical wound braid, helically wound braid, etc.) or woven (e.g., front-back, plain, twill, satin, etc.) together into a single strand. For example, the first plurality of strands 312 may be woven in a front-back pattern weave to create the first strand 310.

According to various embodiments, each of the strands may be interwoven with any or all of the other strands. For example, the fourth strand 340 may be interwoven with the first strand 310 at the first intersection, the second strand 320 at the second intersection, and/or the third strand 330 at the third intersection. In this sense, one or more of the fourth plurality of strands 342 may be the same as the first plurality of strands 312, the second plurality of strands 322, and/or the third plurality of strands 332. In other words, one of the individual strands of the first plurality of strands 312 may be the same as one of the individual strands in any of the other plurality of strands 322, 332, 342, 352, 362. Thus, the first strand 310, the second strand 320, the third strand 330, the fourth strand 340, the fifth strand 350, and the sixth strand 360 may all share one or more individual strands, thereby coupling each of the strands together.

According to various embodiments, the braid or weave pattern may cause the interwoven strands to tighten around one another when the tension in an individual strand is increased. For example, increasing tension in the first strand 310 may cause each of the plurality of strands 312 to tighten around one another. Further, in the example embodiment where one or more of the plurality of strands 312 is interwoven into the fourth strand 340, increasing tension in the first strand 310 may also increase tension in the fourth strand 340 as a result of the individual strands shared by both the first strand 310 and the fourth strand. In this example embodiment, increasing tension in the first strand 310 will increase tension in the fourth strand 340. If the suture assembly is installed into a rounded (e.g., convex) surgical site, increasing the tension in the fourth strand 340 may increase compression on the surgical site that the suture assembly 300 is installed into. According to various embodiments, increasing the compression may facilitate healing of the wound. It should be appreciated that the tension in the fifth strand 350 and the sixth strand 360 may be increased in a similar fashion. Further, increasing the tension in the second strand 320 and/or the third strand 330 may similarly increase the tension in the fourth strand 340, the fifth strand 350, and the sixth strand 360.

According to various embodiments, the second strand 320 is interwoven with the fourth strand 340, the fifth strand 350, and/or the sixth strand 360 such that the tension in the second strand 320 can be increased by increasing the tension in the fourth strand 340, the fifth strand 350, and/or the sixth strand 360. For example, as a part of a rotator cuff repair surgery, the first end 324 and the second end 326 of the second strand 320 may each be coupled to a proximal and distal suture anchor, respectively, implanted in a surgical site. Further, the fourth strand 340, the fifth strand 350, and/or the sixth strand 360 may be coupled to a medial suture anchor implanted in the surgical site. The tension in the second strand 320 may be increased by increasing the tension in the fourth strand 340, the fifth strand 350, and/or the sixth strand 360 as a result of the interwoven strands. For example, the fourth strand 340, the fifth strand 350, and/or the sixth strand 360 may be pulled tighter through the medial anchor(s) to increase the tension of the fourth strand 340, the fifth strand 350, and/or the sixth strand 360, thereby increasing the tension in the second strand 320, which may increase the compressive force applied by the suture assembly 300 to the surgical site.

As shown, the suture assembly 300 further includes a mesh 380 coupled to and extending between the first strand 310, the second strand 320, the third strand 330, the fourth strand 340, the fifth strand 350, and the sixth strand 360. Since the mesh 380 is coupled to each of the strands, the mesh 380 is less likely to bunch up once each of the strands are secured to a suture anchor because the mesh 380 will remain taut once the strands and suture anchors are secured, as is discussed further below. According to various embodiments, the mesh 380 may include a plurality of strands or fibers. For example, the mesh 380 may include a plurality of aligned polymer fibers, such as Poly Lactic-co-Glycolic Acid biodegradable fibers or any other type of biodegradable fibers. According to various embodiments, the mesh comprises collagen polymer fibers. According to various embodiments, the mesh may promote collagen proliferation proximate the surgical site, which may improve healing.

According to various embodiments, the strands or fibers of the mesh 380 may be interwoven into one or more of the first strand 310, the second strand 320, the third strand 330, the fourth strand 340, the fifth strand 350, and the sixth strand 360. In this sense, increasing the tension in any of the strands may also increase the tension in the mesh 380, which may result in greater compression at the surgical site, in a similar manner as described above.

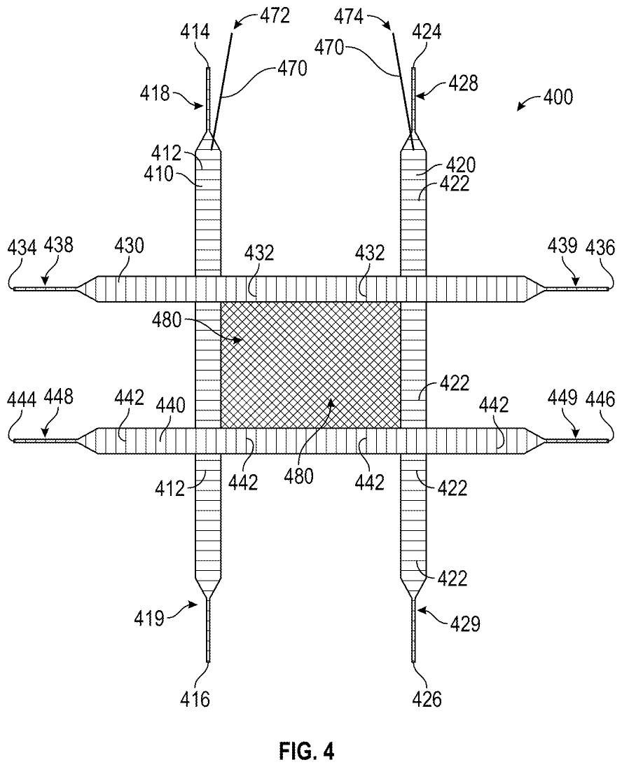

Referring now to FIG. 4, a top view of another suture assembly 400, according to an example embodiment. The suture assembly 400 may be similar to the other suture assemblies 100, 200, 300 described herein. As is described further herein, the suture assembly 400 may be used to secure a portion of tissue in a desired location of a surgical site. As shown, the suture assembly 400 includes a first strand 410, and a second strand 420. As shown, the first strand 410 extends in a first direction and the second strand 420 extends in a second direction, wherein the first direction and the second direction are substantially parallel (e.g., within 2 degrees) with one another. As shown, the first strand 410 includes a first tapered portion 418 proximate a first end 414 and a second tapered portion 419 proximate a second end 416 of the first strand 410. As is discussed further herein, the tapered portions 418, 419 may facilitate coupling the first strand 410 to a suture anchor (e.g., by enabling the first end 414 or the second end 416 to be threaded through a guide loop in a suture anchor). Similarly, the second strand 420 includes a first tapered portion 428 proximate a first end 424 and a second tapered portion 429 proximate a second end 426 of the second strand 420.

The suture assembly 400 further includes a third strand 430 and a fourth strand 440. As shown, the third strand 430 includes a first tapered portion 438 proximate a first end 434 and a second tapered portion 439 proximate a second end 436 of the third strand 430. As is discussed further herein, the tapered portions 438, 439 may facilitate coupling the third strand 430 to a suture anchor (e.g., by enabling the first end 434 or the second end 436 to be threaded through a guide loop in a suture anchor). Similarly, the fourth strand 440 includes a first tapered portion 448 proximate a first end 444 and a second tapered portion 449 proximate a second end 446 of the fourth strand 440.

As shown, the third strand 430 is coupled to the first strand 410 at a first intersection and the second strand 420 at a second intersection. Similarly, the fourth strand 440 is coupled to the first strand 410 at a third intersection and the second strand 420 and a fourth intersection. As shown, the third strand 430 extends in a third direction and the fourth strand 440 extends in a fourth direction, wherein the third direction and the fourth direction are substantially parallel (e.g., within 4 degrees) to one another. Further, the first strand 410 and the second strand 420 are substantially perpendicular (e.g., within 2 degrees) to the third strand 430 and the fourth strand 440 proximate the respective intersections (e.g., the first intersection, the second intersection, etc.).

According to various embodiments, the first strand 410 includes a first plurality of strands 412, the second strand 420 includes a first plurality of strands 422, the third strand 430 may include a third plurality of strands 432, and the fourth strand 440 includes a fourth plurality of strands 442. For example, each of the plurality of strands may include individual strands of absorbable sutures (e.g., gut, Polydioxanone (PDS), Poliglecaprone (MONOCRYL), Polyglactin (Vicryl), etc.) and/or nonabsorbable sutures (e.g., nylon, Polypropylene (Prolene), silk, Polyester (Ethibond), etc.). According to various embodiments, each of the plurality of strands may be braided (e.g., cylindrical wound braid, helically wound braid, etc.) or woven (e.g., front-back, plain, twill, satin, etc.) together into a single strand. For example, the first plurality of strands 412 may be woven in a front-back pattern weave to create the first strand 410.

According to various embodiments, each of the strands may be interwoven with any or all of the other strands. For example, the fourth strand 440 may be interwoven with the first strand 410 at the third intersection and the second strand 420 at the fourth intersection. In this sense, one or more of the fourth plurality of strands 442 may be the same as one or more of the first plurality of strands 412, the second plurality of strands 422, and/or the third plurality of strands 432. In other words, one of the individual strands of the first plurality of strands 412 may be the same as one of the individual strands in any of the other plurality of strands 422, 432, 442. Thus, the first strand 410, the second strand 420, the third strand 430, and the fourth strand 440 may all share one or more individual strands, thereby coupling each of the strands together.

According to various embodiments, the braid or weave pattern may cause the interwoven strands to tighten around one another when the tension in an individual strand is increased. For example, increasing tension in the first strand 410 may cause each of the plurality of strands 412 to tighten around one another. Further, in the example embodiment where one or more of the plurality of strands 412 is interwoven into the third strand 430, increasing tension in the first strand 410 may also increase tension in the fourth strand 440 as a result of the individual strands shared by both the first strand 410 and the fourth strand. In this example embodiment, increasing tension in the first strand 410 will increase tension in the fourth strand 440. If the suture assembly is installed into a rounded (e.g., convex) surgical site, increasing the tension in the fourth strand 440 may increase compression on the surgical site that the suture assembly 400 is installed into. According to various embodiments, increasing the compression may facilitate healing of the wound.

As shown, the suture assembly 400 further includes a mesh 480 coupled to and extending between the first strand 410, the second strand 420, the third strand 430, and the fourth strand 440. Since the mesh 480 is coupled to each of the strands, the mesh 480 is less likely to bunch up once each of the strands are secured to a suture anchor because the mesh 480 will remain taut once the strands and suture anchors are secured, as is discussed further below. According to various embodiments, the mesh 480 may include a plurality of strands or fibers. For example, the mesh 480 may include a plurality of aligned polymer fibers, such as Poly Lactic-co-Glycolic Acid biodegradable fibers or any other type of biodegradable fibers. According to various embodiments, the mesh comprises collagen polymer fibers. According to various embodiments, the mesh may promote collagen proliferation proximate the surgical site, which may improve healing.

According to various embodiments, the strands or fibers of the mesh 480 may be interwoven into one or more of the first strand 410, the second strand 420, the third strand 430, and/or the fourth strand 440. In this sense, increasing the tension in any of the strands may also increase the tension in the mesh 480, which may result in greater compression at the surgical site, in a similar manner as described above.

According to various embodiments, the suture assembly 400 further includes one or more securing strands 470. As is discussed further herein, the securing strand 470 may be secured to one or more suture anchors to increase the compressive force exerted by the suture assembly 400. For example, as shown, the suture assembly 400 includes a securing strand 470 having a first end 472 and a second end 474. As shown, the securing strand 470 is coupled to the remainder of the suture assembly 400. For example, the first strand 410, the second strand 420, and the third strand 430 may be woven such that the each strand defines an inner volume that the securing strand 470 can be threaded through. For example, the first strand 410, the second strand 420, and the third strand 430 may be woven such that the inner volume of each strand is connected. As shown, the securing strand 470 starts at the first end 472, enters the internal volume of the first strand 410, continues through the internal volume of the third strand 430, continues through the internal volume of the second strand 420, and the second end 474 extends out of the second strand 420.

The first end 472 and the second end 474 of the securing strand 470 are configured to be coupled to a suture anchor. For example, the first end 472 may be coupled to the same suture anchor as the first end 414 of the first strand 410 and the second end 474 may be coupled to the same suture anchor as the first end 424 of the second strand 420. Tension in the securing strand 470 may then be increased by pulling the first end 472 and/or the second end 474 through the respective suture anchor. By increasing the tension in the securing strand 470, the tension in the first strand 410, the second strand 420, and the third strand 430 may also increase, thereby increasing the compressive force of the suture assembly 400 (e.g., proximate the point the securing strand 470 is coupled to the suture assembly 400) at the surgical site. It should be appreciated that, while the suture assembly 200 is shown to include just one securing strand 270, other embodiments may include a plurality of securing strand 270. For example, an additional securing strand 470 may be threaded into an internal volume of the first strand 410, the fourth strand 440, and the second strand 420 in a similar fashion.

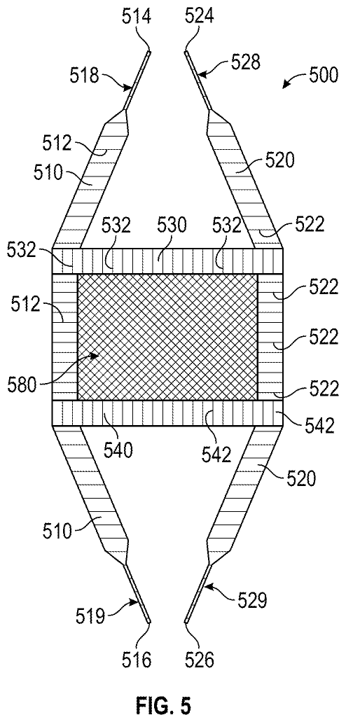

Referring now to FIG. 5, a top view of another suture assembly 500, according to an example embodiment. The suture assembly 500 may be similar to the suture assemblies 100, 200, 300, 400 described above. As is described further herein, the suture assembly 500 may be used to secure a portion of tissue in a desired location of a surgical site. As shown, the suture assembly 500 includes a first strand 510, and a second strand 520. As shown, the first strand 510 extends in a first direction proximate a mesh 580 and the second strand 520 extends in a second direction proximate the mesh 580, wherein the first direction and the second direction are substantially parallel (e.g., within 2 degrees) with one another. Further, the portions of the first strand 510 and the second strand 520 extending away from the mesh are angled towards one another. This allows both the first strand 510 and the second strand 520 to be coupled to the same suture anchor. As shown, the first strand 510 includes a first tapered portion 518 proximate a first end 514 and a second tapered portion 519 proximate a second end 516 of the first strand 510. As is discussed further herein, the tapered portions 518, 519 may facilitate coupling the first strand 510 to a suture anchor (e.g., by enabling the first end 514 or the second end 516 to be threaded through a guide loop in a suture anchor). Similarly, the second strand 520 includes a first tapered portion 528 proximate a first end 524 and a second tapered portion 529 proximate a second end 526 of the second strand 520.

The suture assembly 500 further includes a third strand 530 and a fourth strand 540. As shown, the third strand 530 is coupled to the first strand 510 at a first intersection and the second strand 520 at a second intersection. Similarly, the fourth strand 540 is coupled to the first strand 510 at a third intersection and the second strand 520 and a fourth intersection. As shown, the third strand 530 extends in a third direction and the fourth strand 540 extends in a fourth direction, wherein the third direction and the fourth direction are substantially parallel (e.g., within 2 degrees) to one another. Further, the first strand 510 and the second strand 520 are substantially perpendicular (e.g., within 2 degrees) to the third strand 530 and the fourth strand 540 proximate the respective intersections (e.g., the first intersection, the second intersection, etc.).

According to various embodiments, the first strand 510 includes a first plurality of strands 512, the second strand 520 includes a first plurality of strands 522, the third strand 530 may include a third plurality of strands 532, and the fourth strand 540 includes a fourth plurality of strands 542. For example, each of the plurality of strands may include individual strands of absorbable sutures (e.g., gut, Polydioxanone (PDS), Poliglecaprone (MONOCRYL), Polyglactin (Vicryl), etc.) and/or nonabsorbable sutures (e.g., nylon, Polypropylene (Prolene), silk, Polyester (Ethibond), etc.). According to various embodiments, each of the plurality of strands may be braided (e.g., cylindrical wound braid, helically wound braid, etc.) or woven (e.g., front-back, plain, twill, satin, etc.) together into a single strand. For example, the first plurality of strands 512 may be woven in a front-back pattern weave to create the first strand 510.

According to various embodiments, each of the strands may be interwoven with any or all of the other strands. For example, the fourth strand 540 may be interwoven with the first strand 510 at the third intersection and the second strand 520 at the fourth intersection. In this sense, one or more of the fourth plurality of strands 542 may be the same as one or more of the first plurality of strands 512, the second plurality of strands 522, and/or the third plurality of strands 532. In other words, one of the individual strands of the first plurality of strands 512 may be the same as one of the individual strands in any of the other plurality of strands 522, 532, 542. Thus, the first strand 510, the second strand 520, the third strand 530, and the fourth strand 540 may all share one or more individual strands, thereby coupling each of the strands together.

According to various embodiments, the braid or weave pattern may cause the interwoven strands to tighten around one another when the tension in an individual strand is increased. For example, increasing tension in the first strand 510 may cause each of the plurality of strands 512 to tighten around one another. Further, in the example embodiment where one or more of the plurality of strands 512 is interwoven into the third strand 540, increasing tension in the first strand 510 may also increase tension in the fourth strand 540 as a result of the individual strands shared by both the first strand 510 and the fourth strand. In this example embodiment, increasing tension in the first strand 510 will increase tension in the fourth strand 540. If the suture assembly is installed into a rounded (e.g., convex) surgical site, increasing the tension in the fourth strand 540 may increase compression on the surgical site that the suture assembly 500 is installed into. According to various embodiments, increasing the compression may facilitate healing of the wound.

As shown, the suture assembly 500 further includes a mesh 580 coupled to and extending between the first strand 510, the second strand 520, the third strand 530, and the fourth strand 540. Since the mesh 580 is coupled to each of the strands, the mesh 580 is less likely to bunch up once each of the strands are secured to a suture anchor because the mesh 580 will remain taut once the strands and suture anchors are secured, as is discussed further below. According to various embodiments, the mesh 580 may include a plurality of strands or fibers. For example, the mesh 580 may include a plurality of aligned polymer fibers, such as Poly Lactic-co-Glycolic Acid biodegradable fibers or any other type of biodegradable fibers. According to various embodiments, the mesh comprises collagen polymer fibers. According to various embodiments, the mesh may promote collagen proliferation proximate the surgical site, which may improve healing.

According to various embodiments, the strands or fibers of the mesh 580 may be interwoven into one or more of the first strand 510, the second strand 520, the third strand 530, and/or the fourth strand 540. In this sense, increasing the tension in any of the strands may also increase the tension in the mesh 580, which may result in greater compression at the surgical site, in a similar manner as described above.

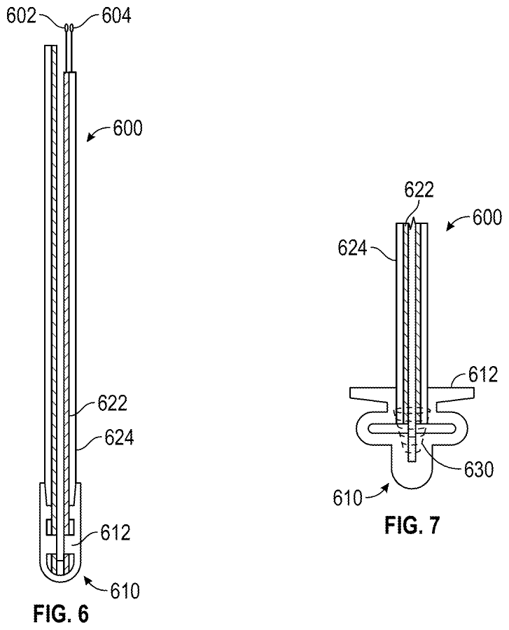

Referring now to FIGS. 6 and 7, cross sectional views of a suture anchor 600 in a first orientation and a second orientation, respectively, are shown according to an example embodiment. The suture anchor 600 may be used to couple a suture assembly to a structure at a surgical site. For example, a suture assembly may be coupled to the suture anchor 600, which may be implanted and fixed within a structure (e.g., a bone). As shown, the suture anchor 600 includes a deformable portion 612 proximate a tip 610 of the suture anchor 600. The deformable portion 612 may be inserted into a bore in the structure in a first orientation (e.g., the orientation shown in FIG. 6). Then, as is described further below, the suture anchor 600 may be set, which causes the deformable portion 612 to deform into a second orientation (e.g., the orientation shown in FIG. 7), such that the suture anchor 600 is prevented from backing out of the bore.

As shown, the suture anchor 600 includes a first thread 622 and a second thread 624. The first thread 622 includes a first guide loop 602 at a first end of the first thread 622. The second thread 624 includes a second guide loop 604 at a first end of the second thread 624. As discussed above, each of the first guide loop 602 and the second guide loop 604 may receive an end of a strand (e.g., a tapered portion of a strand) as a part of coupling the suture assembly to the suture anchor 600. When the end of the strand is inserted into the first guide loop 602 or the second guide loop 604, the second end of the respective thread (e.g., the first thread 622 or the second thread 624) may be pulled, which will cause the respective guide loop to translate towards a tip 610 and eventually though the deformable portion 612, thereby causing the end of the strand to translate through the deformable portion 612. The suture anchor 600 further includes a conical portion 630 within the deformable portion 612. The conical portion 630 is configured to allow the threads 622, 624 and the corresponding ends of the suture assembly to translate in a first direction, while preventing the threads 622, 624 and the corresponding ends of the suture assembly from traveling in a second direction that is opposite the first direction. In other words, once the suture anchor is implanted, the ends of the strands of the suture assembly may be pulled through the suture anchor 600 to increase tension in the strands, while preventing the strands from traveling backwards in the suture anchor 600, which would reduce tension in the strands. It should be appreciated that, while the suture anchor 600 includes a first and second guide loop 602, 604 and therefore can receive two strands of a suture assembly, according to other embodiments, a similar suture anchor may include a different number of guide loops and corresponding threads (e.g., one guide loop, three guide loops, etc.).

It should be appreciated that, while the mesh of the suture assemblies 100, 200, 300, 400, 500 shown in FIGS. 1-5 is generally rectangular, the mesh can be any geometric shape. For example, the mesh may be circular, ovular, triangular, polygonal, or any other shape or abstract design. Further, the strands that are coupled to the mesh may also form any geometric shape when viewed from the top such that the strands surround the mesh. According to various embodiments, the shape of the mesh will depend on the intended use of the suture assembly. For example, the mesh may be shaped to mimic the shape of the recovery area of the surgical site.

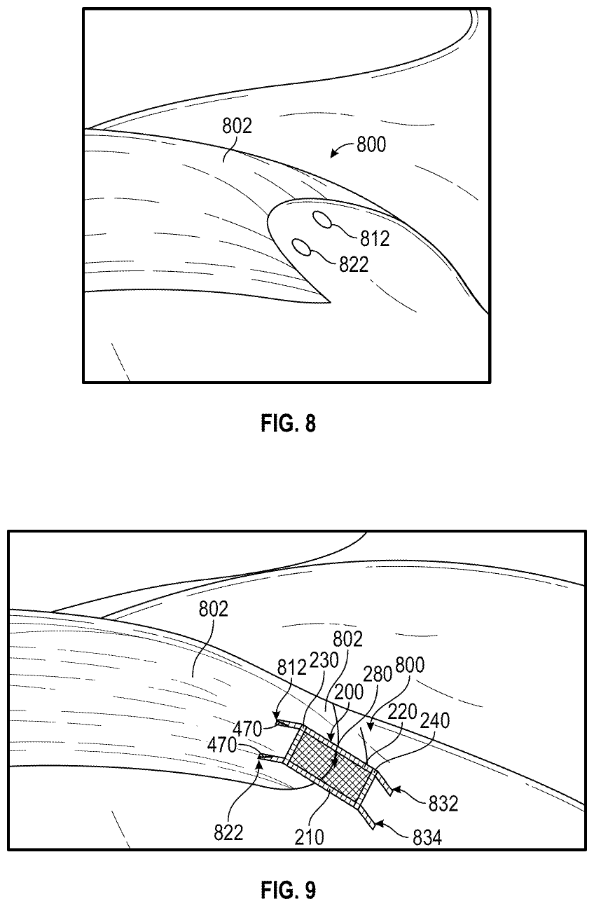

FIG. 8 is a perspective view of a surgical site 800, according to an example embodiment. According to various embodiments, the surgical site 800 may receive a suture assembly to position a portion of tissue 802 in a desired location. As shown, the surgical site 800 includes a first bore 812 and a second bore 822, each of which are configured to receive a suture anchor (e.g., the suture anchor 600).

FIG. 9 is a perspective view of the suture assembly 200 implanted at the surgical site 800. As shown, the tissue 802 was pulled over the first bore 812 and the second bore 822. Suture anchors were then inserted into each of the first bore 812 and the second bore 822. Each of the suture anchors is then coupled to the first strand 210 and the second strand 220 of the suture assembly 200. Further, a third bore 832 and a fourth bore 834 were also created in the structure. Suture anchors (e.g., the suture anchors 600) were then inserted into each of the third bore 832 and the fourth bore 842. Each of the suture anchors is then coupled to the first strand 210 and the second strand 220 of the suture assembly 200. The tension of the first strand 210 and the second strand 220 may then be adjusted by pulling the ends of the first strand 210 and second strand 220 through the respective suture anchor to tighten the first strand 210 and second strand 220. According to various embodiments, increasing the tension in the first strand 210 and the second strand 220 may further increase the tension in the third strand 240 and the fourth strand 260, as is described above. Further, as shown, the mesh 280 is positioned in a desired location over the tissue 802, which may facilitate the tissue 802 healing and reconnecting to the structure.

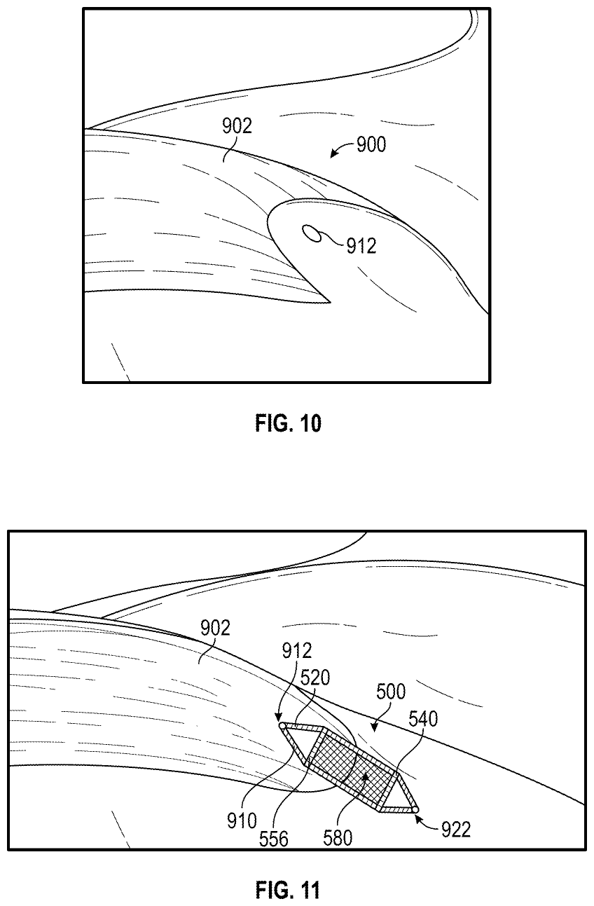

According to various embodiments, the securing strand 270 may also be coupled to the suture assembly 200, the suture anchor at the first bore 812 and the suture anchor at the second bore 822. As discussed above with respect to FIG. 2, a first end 272 of the securing strand 270 may be coupled to the same suture anchor as the first strand 210 and the second end 274 of the securing strand 270 may be coupled to the same suture anchor as the second strand 220. The tension in the securing strand 270 may then be increased (e.g., by pulling the first end 272 and/or the second end 274 though a suture anchor 600 that only allows the securing strand 270 to be pulled through the suture anchor 600 in a single direction), thereby increasing the tension in the third strand 240 and the compressive force of the suture assembly 200 at the surgical site proximate the location at which the securing strand 270 is coupled to the suture assembly 200. According to various embodiments, this may seal the edge (e.g., the portion along the third strand 240) of the suture assembly 200 to the tissue 802. [0082] FIG. 10 is a perspective view of a surgical site 900, according to an example embodiment. According to various embodiments, the surgical site 900 may receive a suture assembly to position a portion of tissue 902 in a desired location. As shown, the surgical site 900 includes a first bore 812, which is configured to receive a suture anchor (e.g., the suture anchor 600).

FIG. 11 is a perspective view of the suture assembly 500 implanted at the surgical site 900. As shown, the tissue 902 was pulled over the first bore 912 and the second bore 922. A suture anchors was inserted into the first bore 912. The suture anchor is then coupled to the first strand 510 and the second strand 520 of the suture assembly 500. Further, a second bore 922 was also created in the structure. A suture anchor (e.g., the suture anchor 600) was then inserted into the second bore 822. The suture anchors is then coupled to the first strand 510 and the second strand 520 of the suture assembly 500. The tension of the first strand 510 and the second strand 520 may then be adjusted by pulling the ends of the strands 510, 520 through the respective suture anchor to tighten the strands 510, 520. According to various embodiments, increasing the tension in the first strand 510 and the second strand 520 may further increase the tension in the third strand 530 and the fourth strand 540, as is described above. Further, as shown, the mesh 580 is positioned in a desired location over the tissue 902, which may facilitate the tissue 902 healing and reconnecting to the structure.

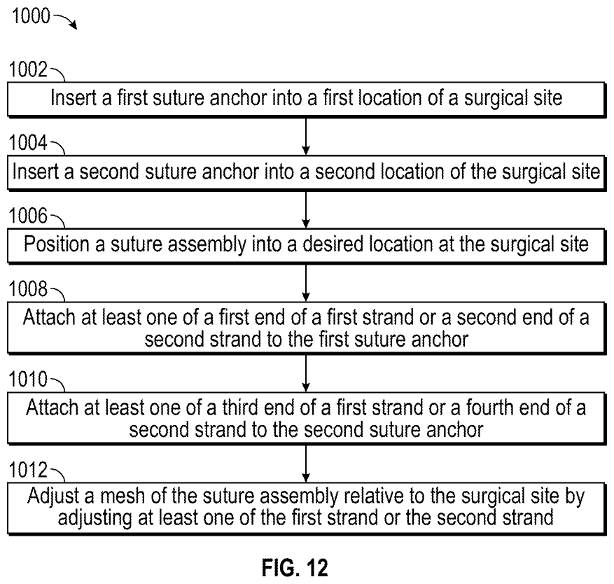

FIG. 12 is a flow diagram of a method of securing tissue 1000, according to an example embodiment. The method of securing tissue 1000 may be utilized, for example, in conjunction with any of the suture assemblies and suture anchors described herein to secure tissue into a desired location (e.g., as shown in FIGS. 9 and 11). For example, the method of securing tissue 1000 may be used to re-attach a muscle (e.g., the bicep, the quadriceps, etc.) or a tendon (e.g., the rotator cuff tendons, the Achilles tendon, etc.). It should be appreciated that the method of securing tissue 1000 need not be performed in the order shown in FIG. 12. Further, some processes shown may be omitted and other processes may be included in the method of securing tissue 1000.

Process 1002 includes inserting a first suture anchor into a first location of a surgical site. For example, the first suture anchor (e.g., the suture anchor 600) may be inserted into a bore in a structure (e.g., the bore 812, the bore 912, etc.).

Process 1004 includes inserting a second suture anchor into a second location of the surgical site. For example, the second suture anchor (e.g., the suture anchor 600) may be inserted into a bore in a structure (e.g., the bore 822, the bore 832, the bore 824, the bore 922, etc.).

Process 1006 includes positioning a suture assembly into a desired location at the surgical site. For example, the suture assembly may include a mesh. Process 1006 may include positioning the mesh over a portion of the tissue (e.g., as shown in FIGS. 9 and 11).

Process 1008 includes attaching at least one of a first end of a first strand or a second end of a second strand to the first suture anchor. For example, the first end of the first strand and/or the second end of the second strand may be inserted into a guide loop of the suture anchor (e.g., the suture anchor 600). The guide loop may then be pulled through a deformable portion (e.g., the deformable portion 612) of the suture anchor, thereby causing the strand of the suture assembly to be pulled through the deformable portion. As discussed above, the suture anchor may prevent the strand of the suture assembly from traveling backwards within the deformable portion, thereby securing the strand to the suture anchor.

Process 1010 includes attaching at least one of a third end of a first strand or a fourth end of a second strand to the second suture anchor. For example, the third end of the first strand and/or the fourth end of the second strand may be inserted into a guide loop of the suture anchor (e.g., the suture anchor 600). The guide loop may then be pulled through a deformable portion (e.g., the deformable portion 612) of the suture anchor, thereby causing the strand of the suture assembly to be pulled through the deformable portion. As discussed above, the suture anchor may prevent the strand of the suture assembly from traveling backwards within the deformable portion, thereby securing the strand to the suture anchor.

Process 1012 includes adjusting a mesh of the suture assembly relative to the surgical site by adjusting at least one of the first strand or the second strand. According to various embodiments, adjusting the mesh includes adjusting the tension in at least one of the first strand and the second strand. For example, the first strand and/or the second strand may continue to be pulled through the suture anchor until a desired tension is achieved in the strand. As discussed above, adjusting the tension in the first strand (e.g., the first strand 210) and/or the second strand (e.g., the second strand 220) may also adjust the tension in a third strand (e.g., the third strand 240), a fourth strand (e.g., the fourth strand 260), and/or a mesh (e.g., the mesh 280). Further, increasing the tension in the first strand and the second strand may additionally increase the compressive force of the mesh onto the surgical site. Once the mesh is finished being adjusted the excess strand extending from the suture anchor may be trimmed.

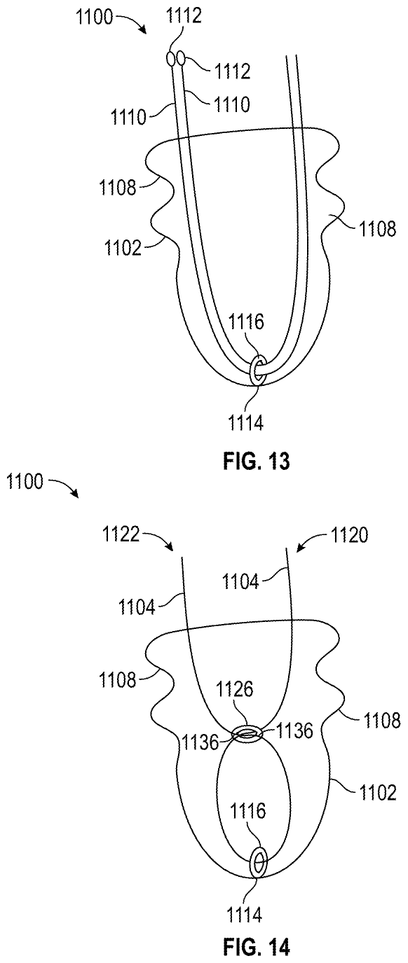

Referring now to FIGS. 13 and 14, a cross sectional view of a suture anchor 1100 is shown, according to an example embodiment. The suture anchor 1100 may share one or more features as any of the other suture anchors described herein (e.g., the suture anchor 600 described above, the suture anchor 1200 described below, etc.). The suture anchor 1100 may further be used in conjunction with one or more of the suture assemblies described herein (e.g., the suture assembly 100, the suture assembly 200, the suture assembly 300, the suture assembly 400, the suture assembly 500, etc.). For example, the suture anchor 1100 may be used to couple a suture assembly to a structure at a surgical site. For example, a suture assembly may be coupled to the suture anchor 1100, which may be implanted and fixed within a structure (e.g., a bone).

As shown, the suture anchor 1100 includes a body portion 1102 that is configured to receive one or more strands (e.g., suture threads) 1104 within an inner volume of the body portion 1102. The body portion 1102 is further configured to be inserted into a desired location at a surgical site. For example, the body portion 1102 may be inserted into a bore (e.g., the bore 812, the bore 822, the bore 832, the bore 842, the bore 912, the bore 922, etc.) to secure a suture assembly in a desired location.

According to various embodiments, the body portion 1102 is configured to prevent backing out of the bore. For example, the outer surface of the body portion 1102 may include one or more ridges 1108 or projections that are configured to allow the suture anchor 1100 to be inserted into a bore and also resist back out movement of the suture anchor 1100. According to various embodiments, the body portion 1102 may be deformable such that the suture anchor 1100 is configured to be inserted into a bore in the structure in a first orientation (e.g., a non-deformed orientation similar to the orientation of the suture anchor 600 shown in FIG. 6). Then, the suture anchor 1100 may be set, which causes the body portion 1102 to deform into a second orientation (e.g., similar to the orientation of the suture anchor 600 shown in FIG. 7), such that the suture anchor 1100 is prevented from backing out of the bore.

As shown in FIG. 13, the suture anchor 1100 includes a first strand 1110 and a second strand 1110 that are configured receive another strand (e.g., as a part of a suture assembly) such that the other strand can be coupled to the suture anchor 1100. The first strand 1110 includes a first guide loop 1112 at a first end of the first strand 1110. The second strand 1110 includes a second guide loop 1112 at a first end of the second strand 1110. As discussed above, each of the guide loops 1112 may individually receive an end of a strand (e.g., a tapered portion of a strand) to couple the suture assembly to the suture anchor 1100. When the end of the strand is inserted into the first guide loop 1112 or the second guide loop 1112, the opposite end of the respective strand may be pulled, which will cause the respective guide loop to translate towards a tip 1114, though a ring 1116 (e.g., a first ring 1116) proximate the tip 1114 and back out the top of the body portion 1102 such that a portion of the strand is strung through the ring 1116 within the internal volume of the body portion 1102, as shown in FIG. 14. While the example embodiment shown in FIG. 13 includes two strands 1110, it should be appreciate that according to other embodiments, any number of strands 1110 may be coupled to the suture anchor 1100 (e.g., one strand 1110, three strands 1110, four strands 1110, etc.). It should further be appreciated that, while the suture anchor 1100 includes a first and second guide loop 1112 and therefore can receive two separate strands of a suture assembly, according to other embodiments, a similar suture anchor 1100 may receive more than one strand within a single guide loop 1112.

According to various embodiments, the body portion 1102 is configured to retain the ring 1116 within the inner volume of the body portion 1102. For example, the ring 1116 may be coupled to the body portion 1102 or integrally formed with the body portion 1102. The ring 1116 may be dimensioned such that the body portion 1102 prevents the ring 1116 from exiting the inner volume of the body portion 1102. According to various embodiments, the ring 1116 may be retained within the inner volume of the body portion 1102 after the body portion 1102 is in the collapsed orientation.

According to various embodiments, the ring 1116 is configured to allow the strands 1110 and/or the strands 1104 to translate through an inner opening of the ring 1116. According to various embodiments, the ring 1116 may be configured to allow the strand to translate both ways through the ring 1116, while according to other embodiments, the ring 1116 may resist movement of a strand in a first direction more than movement of a strand in a second direction that is opposite the first direction. For example, the ring 1116 may include one or more ridges or other texture features that allow the strand 1104 to travel through the ring 1116 but resist movement of the strand 1104 back out of the ring 1116, which may help prevent the suture assembly from loosening after the suture anchor 1100 is installed.

Referring now to FIG. 14, the suture anchor 1100 is shown coupled to a strand 1104. For example, a first end 1120 of the strand 1104 may be stranded through a guide loop 1112 (see FIG. 13) such that the corresponding strand 1110 may be pulled through the ring 1116 such that the strand 1104 is stranded through the ring 1116. According to various embodiments, the strand 1104 is a part of a suture assembly, such as one of the suture assemblies described herein.

As shown, the strand 1104 is coupled to a second ring 1126. For example, the second ring 1126 may be coupled to or integrally formed with a part of a suture assembly. According to various embodiments, the second ring 1126 is coupled to a portion 1136 of the strand 1104 such the second ring 1126 does not translate along the strand 1104 proximate the portion 1136. Further, the second ring 1126 is configured to allow the first end 1120 of the strand 1104 to pass through a center opening of the second ring 1126. For example, as shown, the second end 1122 is stranded through the first ring 1116, through the second ring 1126, and out of the top of the suture anchor 1100. According to various embodiments, the second ring 1126 may allow translation of the strand 1104 through the center opening of the second ring in a first direction while resisting movement of the strand 1104 in a second direction that is opposite the first direction in a similar manner as the first ring 1116 described above.

According to various embodiments, the arrangement shown in FIG. 14 is configured to allow tightening of the suture assembly that is coupled to the suture anchor 1100 while also preventing unwanted loosening of the suture assembly. For example, after the suture anchor 1100 is coupled to a suture assembly and installed into a structure at a surgical site, a second end 1122 of the strand 1104 may be pulled to tighten the suture assembly. For example, pulling the second end 1122 may cause the first end 1120 to translate towards the tip 1114 of the suture assembly while simultaneously causing the second ring 1126 to move away from the tip 1114 to increase tension in the strand 1104. Once a desired tension is achieved in the strand 1104, the second end 1122 may be trimmed and the arrangement of the first ring 1116 and the second ring 1126 will help prevent a significant reduction in the tension in the strand 1104.

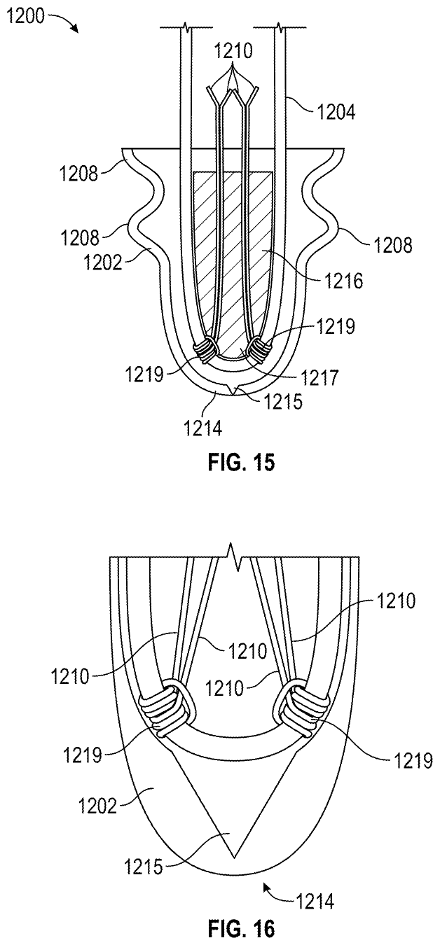

Referring now to FIGS. 15 and 16, a cross sectional view of a suture anchor 1200 is shown, according to an example embodiment. The suture anchor 1200 may share one or more features as any of the other suture anchors described herein (e.g., the suture anchor 600, the suture anchor 1100, etc.). The suture anchor 1200 may further be used in conjunction with one or more of the suture assemblies described herein (e.g., the suture assembly 100, the suture assembly 200, the suture assembly 300, the suture assembly 400, the suture assembly 500, etc.). For example, the suture anchor 1200 may be used to couple a suture assembly to a structure at a surgical site. For example, a suture assembly may be coupled to the suture anchor 1200, which may be implanted and fixed within a structure (e.g., a bone).

As shown, the suture anchor 1200 includes a body portion 1202 that is configured to receive a main strand 1204 and/or one or more strands (e.g., suture strands) 1210 within an inner volume of the body portion 1202. The body portion 1202 is further configured to be inserted into a desired location at a surgical site. For example, the body portion 1202 may be inserted into a bore (e.g., the bore 812, the bore 822, the bore 832, the bore 842, the bore 912, the bore 922, etc.) to secure a suture assembly in a desired location.

According to various embodiments, the body portion 1202 is configured to prevent backing out of the bore. For example, the outer surface of the body portion 1202 may include one or more ridges 1208 or projections that are configured to allow the suture anchor 1200 to be inserted into a bore and also resist back out movement of the suture anchor 1200. According to various embodiments, the body portion 1202 may be deformable such that the suture anchor 1200 is configured to be inserted into a bore in the structure in a first orientation (e.g., a non-deformed orientation similar to the orientation of the suture anchor 600 shown in FIG. 6). Then, the suture anchor 1200 may be set, which causes the body portion 1202 to deform into a second orientation (e.g., similar to the orientation of the suture anchor 600 shown in FIG. 7), such that the suture anchor 1200 is prevented from backing out of the bore. According to various embodiments, setting the suture anchor 1202 causes an inner portion 1216 of the body portion 1202 to translate towards a tip 1214 of the suture anchor 1200. When set, the middle portion 1216 may restrict translation of the main strand 1204. For example, a tip 1217 of the middle portion 1216 maybe be received within a slot 1215 of the anchor body 1202 and the main strand 1204 may be pinched there between to restrict translation of the main strand 1204 within the body portion 1202.

As shown, a plurality of strands 1210 are coupled to the main strand 1204. The plurality of strands 1210 may be coupled to or integrally formed with a part of a suture assembly, such as one of the suture assemblies described herein. As is discussed further below, the strands 1210 are coupled to the main strand 1204 via a non-slipping knot 1219 (e.g., a prussik knot, a prussic hitch, etc.). In this sense, once the main strand 1204 is fixed within the suture anchor 1200 (e.g., after setting the suture anchor 1200), the strands 1210 are prevented from translating along the main strand 1204 in response to a pulling force or tension on the strands 1210, thereby keeping the corresponding suture assembly in tension at the desired location. While the example embodiment shown in FIGS. 15 and 16 includes two separate strands 1210 coupled to the main strand 1204, resulting in four separate ends, it should be appreciate that according to other embodiments, any number of strands 1210 may be coupled to the suture anchor 1200 (e.g., one strand 1210, three strands 1210, four strands 1210, etc.). It should further be appreciated that, while the suture anchor 1100 includes a first and second guide loop 1112 and therefore can receive two separate strands of a suture assembly, according to other embodiments, a similar suture anchor 1100 may receive more than one strand within a single guide loop 1112.

According to various embodiments, the arrangement shown in FIGS. 15 and 16 is configured to allow tightening of the suture assembly that is coupled to the suture anchor 1200 while also preventing unwanted loosening of the suture assembly. For example, before the anchor is set, one or more strands 1210 of a suture assembly can be coupled to the main strand 1204 via a non-slipping knot 1219. Once the strands 1210 are coupled to the main strand 1204, the main strand can be stranded into the suture anchor 1200 and pulled tight until a desired tension is achieved in each of the strands 1210. Then, the suture anchor 1200 may be set to fix the position of the main strand 1204, thereby preventing a significant reduction in the tension in the strands 1210.

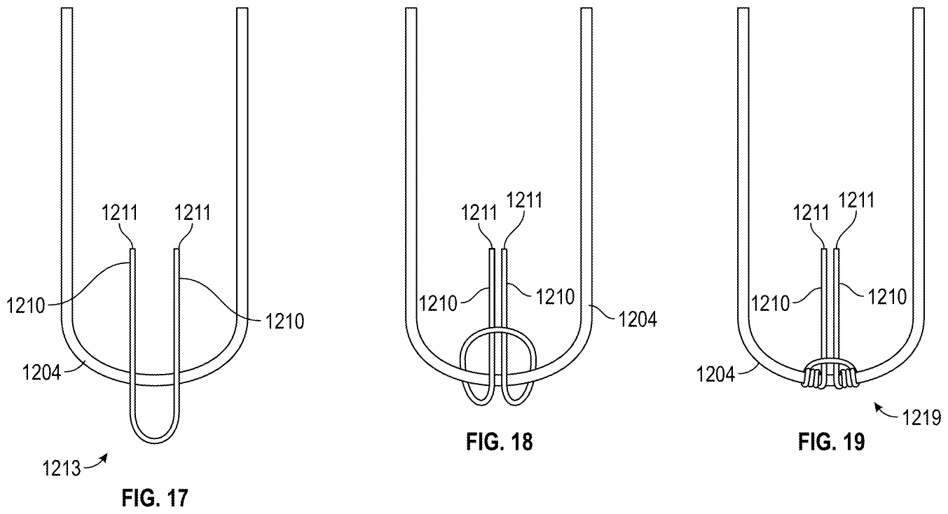

Referring now to FIGS. 17-19, the process of tying a non-slipping knot (e.g., a prussik knot, a prussic hitch, etc.) for use within the suture anchor 1200 of FIG. 15 is shown, according to an example embodiment. As shown in FIG. 17, the strand 1210 is bent into a partial loop 1213 (e.g., a U-shape) and placed over the main strand 1204 such that the free ends 1211 of the strand 1200 are on one side of the main strand 1204 and the partial loop 1213 is on the opposite side of the main strand 1204. As shown in FIG. 18, the loose ends 1211 are wrapped around the main strand 1204 and pulled through the partial loop 1213. As shown in FIG. 19, the loose ends 1211 are wrapped around the main strand 1204 a second time, pulled through the partial loop 1213 a second time, and pulled right. This knot will prevent the strand 1210 from translating along the main strand 1204 as a result of a pulling force being applied to one of the loose ends 1211.

As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. The devices, systems and methods described herein may be embodied in other specific forms without departing from the characteristics thereof. The foregoing implementations are illustrative rather than limiting of the described systems and methods. The scope of the systems and methods described herein is thus indicated by the appended claims, rather than the foregoing description, and changes that come within the meaning and range of equivalency of the claims are embraced therein.

What is claimed is:

1. A suture assembly comprising:
a first strand having a first end, and a second end, the first strand configured to be secured to one or more suture anchors;
a second strand having a third end, a fourth end, the second strand configured to be secured to the one or more suture anchors;
a third strand coupled to first strand at a first intersection and the second strand at a second intersection;
a fourth strand coupled to the first strand at a third intersection and the second strand at a fourth intersection; and
a mesh attached to and extending between the first strand, the second strand, the third strand and the fourth strand.

2. The suture assembly of claim 1, wherein the first strand comprises a first tapered portion proximate the first end and a second tapered portion proximate the second end.

3. The suture assembly of claim 1, wherein the first strand is substantially perpendicular to the third strand proximate the first intersection and the second strand is substantially perpendicular to the second strand proximate the second intersection.

4. The suture assembly of claim 1, wherein the mesh comprises suture tape.

5. The suture assembly of claim 1, wherein the mesh comprises aligned polymer fibers.

6. The suture assembly of claim 1, wherein the first strand comprises a first plurality of strands, the second strand comprises a second plurality of strands, the third strand includes a third plurality of strands, and the fourth strand includes a fourth plurality of strands;
wherein the first plurality of strands is interwoven with the third plurality of strands and the fourth plurality of strands; and
wherein the second plurality of strands is interwoven with the third plurality of strands and the fourth plurality of strands.

7. The suture assembly of claim 6, wherein the first plurality of strands is woven in a front-back orientation.

8. The suture assembly of claim 1, wherein the one or more suture anchors includes a first suture anchor having a first ring positioned within the suture anchor and configured to receive a first end of the first strand such that a first portion of the first strand translates through the first ring; and
wherein a second ring is coupled to a second portion of the first strand such that the second portion is fixed relative to the second ring, the second ring configured to receive the first end of the first strand.

9. The suture assembly of claim 1, further comprising a main strand configured to be coupled the first strand via a non-slipping knot, wherein the main strand is configured to be secured within at least one of the one or more suture anchors.

10. The suture assembly of claim 1, wherein the first strand extends in a first direction between the first intersection and the third intersection and the third strand extends in a second direction between the first intersection and the second intersection; and
wherein increasing a first tension in the first direction in the first strand causes an increase in a second tension in the second direction in the second strand.

11. A method of securing tissue, the method comprising:
inserting a first suture anchor at a first location of a surgical site and a second suture anchor at a second location of the surgical site;
positioning a suture assembly at the surgical site, the suture assembly comprising a first strand, a second strand, a third strand, a fourth strand, and a mesh attached to and extending between the first strand, the second strand, the third strand and the fourth strand, wherein the third strand and the fourth strand are adjustably engaged with a first portion and a second portion of the first strand, respectively, and the third strand and the fourth strand are adjustably engaged with a third portion and a fourth portion of the second strand, respectively;
attaching at least one of a first end of the first strand or a third end of the second strand to the first suture anchor;
attaching at least one of a second end of the first strand or a fourth end of the second strand to the second suture anchor; and
adjusting the mesh relative to the surgical site by:
adjusting at least one of a first tension in the first strand or a second tension in the third strand to cause a change in a third tension in the third strand and a fourth tension in the fourth strand.

12. The method of claim 11, further comprising:
inserting a third suture anchor at a third location of the surgical site; and
attaching at least one of the first end of the first strand or the third end of the second strand to the third suture anchor.

13. The method of claim 11, further comprising:
inserting a fourth suture anchor at a fourth location of the surgical site; and
attaching at least one of the second end of the first strand or the fourth end of the second strand to the fourth suture anchor.

14. The method of claim 11, wherein the first strand comprises a first tapered portion proximate the first end and a second tapered portion proximate the second end.

15. The method of claim 14, wherein coupling the first end of the first strand to the first suture anchor comprises inserting the first tapered portion into a guide loop in the first suture anchor.

16. The method of claim 11, wherein the first strand and the second strand are substantially perpendicular to the third strand and the fourth strand proximate the mesh.

17. The method of claim 11, wherein the mesh comprises suture tape.

18. The method of claim 11, wherein the mesh comprises aligned polymer fibers.

19. The method claim 11, wherein the first strand comprises a first plurality of strands, the second strand comprises a second plurality of strands, the third strand includes a third plurality of strands, and the fourth strand includes a fourth plurality of strands;
wherein the first plurality of strands is interwoven with the third plurality of strands and the fourth plurality of strands; and
wherein the second plurality of strands is interwoven with the third plurality of strands and the fourth plurality of strands.

20. The method of claim 19, wherein the first plurality of strands is woven in a front-back orientation.

21. A surgical kit, comprising:
a first suture anchor;
a second suture anchor; and
a suture assembly configured to be coupled to the first suture anchor and the second suture anchor, the suture assembly comprising:
a first strand having a first end, and a second end, the first strand configured to be secured to one or more suture anchors;
a second strand having a third end, a fourth end, the second strand configured to be secured to the one or more suture anchors;
a third strand coupled to first strand at a first intersection and the second strand at a second intersection;
a fourth strand coupled to the first strand at a third intersection and the second strand at a fourth intersection; and
a mesh attached to and extending between the first strand, the second strand, the third strand and the fourth strand.

22. The surgical kit of claim 21, wherein the first strand comprises a first tapered portion proximate the first end and a second tapered portion proximate the second end.

23. The surgical kit of claim 21, wherein the first strand and the second strand are substantially perpendicular to the third strand and the fourth strand proximate the mesh.

24. The surgical kit of claim 21, wherein the mesh comprises suture tape.

25. The surgical kit of claim 21, wherein the mesh comprises aligned polymer fibers.

26. The surgical kit of claim 21, wherein the first suture anchor and the second suture anchor are knotless self-punching shuttle anchors.

27. The surgical kit of claim 21, wherein the first strand comprises a first plurality of strands, the second strand comprises a second plurality of strands, the third strand includes a third plurality of strands, and the fourth strand includes a fourth plurality of strands;
wherein the first plurality of strands is interwoven with the third plurality of strands and the fourth plurality of strands; and
wherein the second plurality of strands is interwoven with the third plurality of strands and the fourth plurality of strands.

28. The surgical kit of claim 27, wherein the first plurality of strands is woven in a front-back orientation.

29. The surgical kit of claim 21, wherein the first strand extends in a first direction between the first intersection and the third intersection and the third strand extends in a second direction between the first intersection and the second intersection; and
wherein increasing a first tension in the first direction in the first strand causes an increase in a second tension in the second direction.
